(19) United States
(12) Patent Application Publication
(10) Pub. No.: US 2009/0253983 A1
(43) Pub. Date: Oct. 8, 2009

(54) IMAGE BASED MEASUREMENT OF CONTRAST AGENTS

(75) Inventors: Thomas Kwok-Fah Foo, Clifton Park, NY (US); Daniel James Blezek, Rochester, MN (US); William Thomas Dixon, Clifton Park, NY (US)

Correspondence Address: GENERAL ELECTRIC COMPANY (PCPI);C/O FLETCHER YODER, P. O. BOX 692289, HOUSTON, TX, 77269-2289, US

(73) Assignee: General Electric Company, Schenectady, NY (US)

(21) Appl. No.: 12/099,006

(22) Filed: Apr. 7, 2008

(51) Int. Cl. A61B 5/055

(52) U.S. Cl. 600/420; 600/431

(57) ABSTRACT

Provided is a method, including accessing or acquiring a phase image including a contrast agent enhanced region and determining a quantity of a contrast agent based on the phase image. Further provided is a computer program for determining the quantity of a substance in a region. The program is constructed and arranged to access or acquire a contrast enhanced image, to execute a fitting algorithm based on data contained in the contrast enhanced image, and to determine the quantity of the substance based on the output/result/outcome of the fitting algorithm.

### BACKGROUND

The invention relates generally to nuclear magnetic resonance imaging (“MRI”), and more particularly to a technique for estimating a quantity of a substance (e.g., a contrast agent) contained in a region.

MRI systems have become ubiquitous in the field of medical diagnostics. Such systems are used to produce magnetic resonance (MR) images of a person's anatomy. In general, MRI systems are based on the interactions among a primary magnetic field, a radio frequency (RF) field, and time varying magnetic gradient fields with the subject of interest. Certain nuclear components, such as hydrogen nuclei in water molecules found in a patient, have characteristic behaviors in response to the external magnetic fields generated by the MRI system. One response includes the spin of certain nuclear components in varying relations to one another. The precession of spins of such nuclear components can be influenced by manipulation of the magnetic fields to generate RF signals that are indicative of the responses and that can be detected, processed, and used to reconstruct a useful image.

The magnetic fields used to produce MR images include a highly uniform, static magnetic field that is produced by a primary magnet. A series of gradient fields are produced by a set of three gradient coils disposed around the subject. The gradient fields encode positions of individual volume elements, or voxels, in three dimensions. A radiofrequency coil is employed to produce an RF magnetic field, typically pulsed to create the required resonance signals. This RF magnetic field perturbs the spins from their equilibrium direction, causing the spins to precess at desired phases and frequencies. During this precession, RF fields are emitted by the spins and detected by either the same transmitting RF coil, or by a separate receive-only coil. These signals are amplified, filtered, and digitized. The digitized signals are then processed using one of several possible reconstruction algorithms to reconstruct a useful image.

To enhance the image, a contrast agent can be administered to the subject to delineate certain areas of interest. The contrast agent generally includes water, a paramagnetic compound, a super paramagnetic compound, or a similar substance that can be detected within the subject. Specifically, the contrast agent may modify the characteristics (e.g., relaxation time) of certain nuclear components, thereby providing enhanced contrast within the image. One example of a contrast agent includes supermagnetic iron oxide (SPIO) particles. SPIO particles include small particles of ferrite that exhibit strong relaxation properties in their vicinity and that help to enhance the contrast of their surroundings (due to increase of magnetic susceptibility). Another exemplary contrast agent includes gadolinium-DTPA (diethylenetriaminepentacetic acid).

In certain procedures, the contrast agents accumulate in specific regions of interest. For example, a contrast agent that is attracted to a specific organ within the body can be administered to a patient to enhance the contrast of the organ within the image. Although observing the region with the contrast agent may be helpful, it may be desirable to determine the amount (e.g., quantity) of a contrast agent located within a specific region. However, estimations of the concentration or the quantity (e.g., amount) of the contrast agent may be imprecise and lead to inaccurate results.

Accordingly, there is a need for an improved technique for determining the quantity of a substance that is located in a specific region. Particularly, there is a need for a technique that provides for more accurately determining the quantity of a contrast agent in a region based on an MR image.

### BRIEF DESCRIPTION

The present technique provides a novel method and system for determining the amount of a substance contained within a region. In accordance with one embodiment of the present technique, provided is a method, including accessing or acquiring a phase image that includes a contrast agent enhanced region and determining a quantity of a contrast agent based on the phase image.

In accordance with another embodiment of the present technique, provided is a method of estimating a quantity of a contrast agent. The method includes constructing a phase difference image based on first and second images, unwrapping the phase difference image to generate an unwrapped phase difference image, designating a dipole center of a depicted contrast agent in the unwrapped phase difference image, designating one or more surrounding regions of interest, wherein the one or more surrounding regions of interest do not overlap the depicted contrast agent, and estimating the quantity of the contrast agent based on the identified dipole center and the one or more surrounding regions of interest.

In accordance with another embodiment of the present technique, provided is a method that includes estimating a quantity of a contrast agent depicted in an MR image based on the phase gradient values observed in a region of the MR image that surrounds the depicted contrast agent.

In accordance with another embodiment of the present technique, provided is a computer readable medium storing a computer program for determining the quantity of a substance in a region. The program is constructed and arranged to access or acquire a contrast enhanced image, to execute a fitting algorithm based on data contained in the contrast enhanced image, and to determine the quantity of the substance based on the output/result/outcome of the fitting algorithm.

### DRAWINGS

These and other features, aspects, and advantages of the present invention will become better understood when the following detailed description is read with reference to the accompanying drawings in which like characters represent like parts throughout the drawings, wherein:

FIG. 1 is a diagrammatical representation of an MRI system for use in medical diagnostics in accordance with certain embodiments of the present technique;

FIG. 2 is a block diagram illustrating a method of determining a quantity of a contrast agent in accordance with certain embodiments of the present technique;

FIG. 3 is a block diagram illustrating an alternate embodiment of a method of determining a quantity of a contrast agent in accordance with certain aspects of the present technique;

FIG. 4A is an illustration of an exemplary dipole magnetic field pattern in accordance in accordance with certain aspects of the present technique;

FIG. 4B is an exemplary phase image in accordance in accordance with certain aspects of the present technique;

FIG. 4C is an exemplary phase difference image in accordance in accordance with certain aspects of the present technique;

FIG. 5 is an exemplary contrast enhanced image in accordance in accordance with certain aspects of the present technique; and

FIG. 6 is a plot illustrating exemplary results of quantity determinations in accordance with certain aspects of the present technique.

### DETAILED DESCRIPTION

The embodiments discussed below provide a technique for determining the amount (e.g., quantity) of a substance (e.g., a contrast agent) depicted in an image acquired by a magnetic resonance imaging (MRI) system. In certain embodiments, the technique includes acquiring one or more contrast enhanced MR images (e.g., phase images) that include a particular region or location that is indicative of the substance (e.g., the contrast agent), and employing one or more image processing techniques to determine the quantity of the substance based on the location of the substance and its surrounding features (e.g., phase gradient).

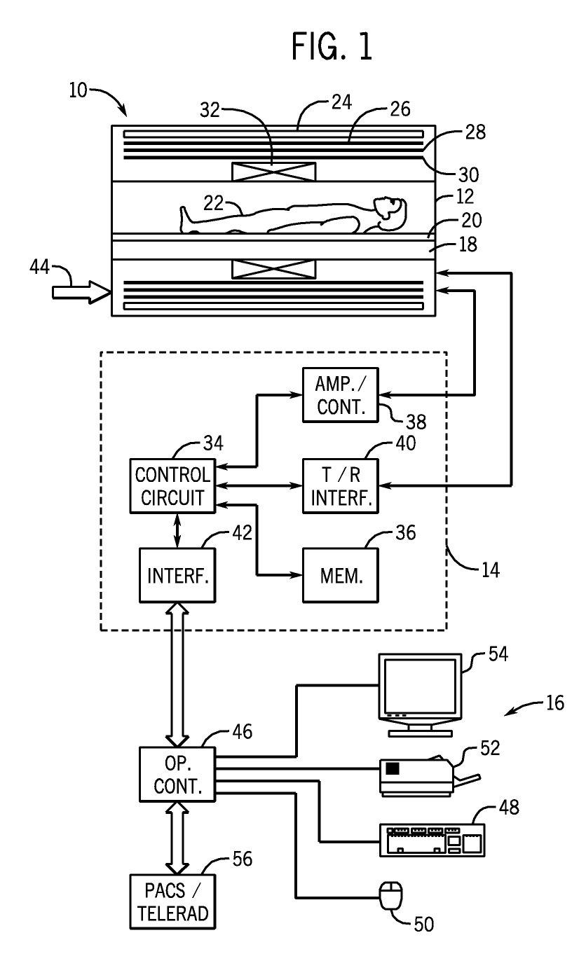

Turning now to the figures, and referring first to FIG. 1, a magnetic resonance imaging (MRI) system 10 suitable for MR imaging is illustrated diagrammatically as including a scanner 12, scanner control circuitry 14, and system control circuitry 16. While the MRI system 10 may include any suitable MRI scanner or detector, in the illustrated embodiment the system 10 includes a full body scanner comprising a patient bore 18 into which a table 20 may be positioned to place a patient 22 in a desired position for scanning.

The scanner 12 may be of any suitable type of rating, including scanners varying from 0.5 Tesla ratings to 1.5 Tesla ratings and beyond. The scanner 12 includes a series of associated coils for producing controlled magnetic fields, for generating RF excitation pulses, and for sensing emissions from gyromagnetic material within the patient in response to such pulses. In the diagrammatical view of FIG. 1, a primary magnet coil 24 is provided for generating a primary magnetic field generally aligned with the patient bore 18. A series of gradient coils 26, 28, and 30 are grouped in a coil assembly for generating controlled magnetic gradient fields during examination sequences as described more fully below. An RF coil 32 is provided for generating RF pulses for exciting gyromagnetic material present in the patient 22. In the embodiment illustrated in FIG. 1, the RF coil 32 also serves as a receiving coil. Thus, the RF coil 32 may be coupled with driving and receiving circuitry in passive and active modes for receiving emissions from the gyromagnetic material and for applying RF excitation pulses, respectively. Alternatively, various configurations of receiving coils may be provided separate from the RF coil 32. Such coils may include structures specifically adapted for target anatomies, such as head coil assemblies, and so forth. Moreover, receiving coils may be provided in any suitable physical configuration, including phased array coils, and so forth.

In a present configuration, the gradient coils 26, 28 and 30 have different physical configurations adapted to their function in the imaging system 10. As will be appreciated by those skilled in the art, the coils are comprised of conductive wires, bars or plates which are wound or cut to form a coil structure which generates a gradient field upon application of controlled pulses as described below. The placement of the coils within the gradient coil assembly may be done in several different orders, but in the present embodiment, a Z-axis coil is positioned at an innermost location, and is formed generally as a solenoid-like structure, which has relatively little impact on the RF magnetic field. Thus, in the illustrated embodiment, gradient coil 30 is the Z-axis solenoid coil, while coils 26 and 28 are Y-axis and X-axis coils respectively. The coils of the scanner 12 are controlled by external circuitry to generate desired fields and pulses, and to read signals from the gyromagnetic material in a controlled manner.

As will be appreciated by those skilled in the art, when the material, typically bound in tissues of the patient 22, is subjected to the primary field, individual magnetic moments of the paramagnetic nuclei in the tissue partially align with the field. While a net magnetic moment is produced in the direction of the polarizing field, the randomly oriented components of the moment in a perpendicular plane generally cancel one another. During an examination sequence, an RF frequency pulse is generated at or near the Larmor frequency of the nuclei of interest, resulting in rotation of the net aligned moment to produce a net transverse magnetic moment. This transverse magnetic moment precesses around the main magnetic field direction, emitting RF (magnetic resonance) signals. Different molecules within the patient 22 generally have different responses to the magnetic fields, and the emitted RF signals are typically indicative of the differences. For reconstruction of the desired images, these RF signals are detected by scanner 12 and processed. As described more fully below, contrast agents may be administered to a patient to enhance the contrast of various regions of a patient's anatomy. Typically, in the presence of contrast agents, the nuclei of interest responds to the magnetic fields and RF signals in a manner that makes them more visible within the resulting image. For example, a region containing the contrast agent may appear as a hypointense or hyperintense region (e.g., light or dark spot) in the resulting MR image.

Gradient coils 26, 28, and 30 serve to generate precisely controlled magnetic fields, the strength of which vary over a predefined field of view, typically with positive and negative polarity. When each coil is energized with known electric current, the resulting magnetic field gradient is superimposed over the primary field and produces a desirably linear variation in the Z-axis component of the magnetic field strength across the field of view. The field generated by each respective gradient coil 26, 28, 30 varies linearly in one direction, but is homogenous in the other two. The three coils have mutually orthogonal axes for the direction of their variation, enabling a linear field gradient to be imposed in an arbitrary direction with an appropriate combination of the three gradient coils 26, 28, and 30.

The pulsed gradient fields perform various functions integral to the imaging processes. For example, the gradient pulses may be applied to produce a gradient recalled echo pulse. As discussed in more detail below, the polarity of each gradient pulse may be varied with each successive RF pulse. For imaging, some of these functions are slice selection, frequency encoding and phase encoding. These functions can be applied along the X-, Y- and Z-axis of the original physical coordinate system or in various arbitrary physical directions determined by combinations of pulsed currents applied to the individual gradient coils.

The slice select gradient determines a slab of tissue or anatomy to be imaged in the patient. The slice select gradient field may be applied simultaneously with a frequency selective RF pulse to excite a known volume of spins within a desired slice that precess at the same frequency. The slice thickness is determined by the bandwidth of the RF pulse and the gradient strength across the field of view.

The frequency encoding gradient is also known as the readout gradient, and is usually applied in a direction perpendicular to the slice select gradient. In general, the frequency encoding gradient is applied before and during the formation of the MR echo signal resulting from the RF excitation. Spins of the gyromagnetic material under the influence of this gradient are frequency encoded according to their spatial position along the gradient field. By Fourier transformation, acquired signals may be analyzed to identify their location in the selected slice by virtue of the frequency encoding.

Finally, the phase encode gradient is generally applied before the frequency encoding gradient and after the slice select gradient. Localization of spins in the gyromagnetic material in the phase encode direction is accomplished by sequentially inducing variations in phase of the precessing protons or nuclei of the material using slightly different gradient amplitudes that are applied in a known order during the data acquisition sequence. The phase encode gradient permits phase differences to be created among the spins of the material in accordance with their position in the phase encode direction.

As will be appreciated by those skilled in the art, a great number of variations may be devised for pulse sequences employing the exemplary gradient pulse functions described above as well as other gradient pulse functions not explicitly described here. Moreover, adaptations in the pulse sequences may be made to appropriately orient both the selected slice and the frequency and phase encoding to excite the desired material and to acquire resulting MR signals for processing.

The coils of scanner 12 are controlled by scanner control circuitry 14 to generate the desired magnetic field and RF pulses. In the diagrammatical view of FIG. 1, control circuitry 14 thus includes a control circuit 34 for commanding the pulse sequences employed during the examinations, and for processing received signals. Control circuit 34 may include any suitable programmable logic device, such as a CPU or digital signal processor of a general purpose or application-specific computer. Control circuit 34 further includes memory circuitry 36, such as volatile and/or non-volatile memory devices for storing physical and logical axis configuration parameters, examination pulse sequence descriptions, acquired image data, programming routines, and so forth, used during the examination sequences implemented by the scanner.

Interface between the control circuit 34 and the coils of scanner 12 are managed by amplification and control circuitry 38 and by transmission and receive interface circuitry 40. Circuitry 38 includes amplifiers for each gradient field coil to supply drive current to the field coils in response to control signals from control circuit 34. Interface circuitry 40 includes additional amplification circuitry for driving RF coil 32. Moreover, where the RF coil 32 serves both to emit the RF excitation pulses and to receive MR signals, circuitry 38 will typically include a switching device for toggling the RF coil between active or transmitting mode, and passive or receiving mode. Finally, control circuitry 14 includes interface components 42 for exchanging configuration and image data with system control circuitry 16. A power supply, denoted generally by reference numeral 44 in FIG. 1, is provided for energizing the primary coil 24.

System control circuitry 16 may include a wide range of devices for facilitating interface between an operator or radiologist and scanner 12 via scanner control circuitry 14. In the illustrated embodiment, for example, an operator controller 46 is provided in the form of a computer work station employing a general purpose or application-specific computer. The station also typically includes memory circuitry for storing examination pulse sequence descriptions, examination protocols, user and patient data, image data, both raw and processed, and so forth. The station may further include various interface and peripheral drivers for receiving and exchanging data with local and remote devices. In the illustrated embodiment, such devices include a conventional computer keyboard 48 and an alternative input device such as a mouse 50. A printer 52 is provided for generating hard copy output of documents and images reconstructed from the acquired data. A computer monitor 54 is provided for facilitating operator interface. In addition, system 10 may include various local and remote image access and examination control devices, represented generally by reference numeral 56 in FIG. 1. Such devices may include picture archiving and communication systems (Pacs), teleradiology systems (Telerad), and so forth.

It should be noted that, while in the present description reference is made to a horizontal cylindrical bore imaging system employing a superconducting primary field magnet assembly, the present technique may be applied to various other configurations, such as scanners employing vertical fields generated by superconducting magnets, permanent magnets, electromagnets or combinations of these means. Additionally, while FIG. 1 generally illustrates an exemplary closed MRI system, the embodiments of the present invention are applicable in open MRI systems that are designed to allow access by a physician.

As briefly discussed above, contrast agents may be administered to delineate certain areas of interest within the anatomy of Patient 22. The contrast agent may include water, a paramagnetic compound, a super-paramagnetic compound, or a similar substance that is detectable by the MRI system. Specifically, the contrast agent may modify the characteristics (e.g., relaxation time) of surrounding molecules, thereby providing enhanced contrast within the image. One example of a contrast agent includes supermagnetic iron oxide (SPIO) particles. SPIO particles include small particles of ferrite that exhibit strong relaxation properties that help to enhance the contrast from their surroundings. Another example includes gadolinium-DTPA (diethylenetriaminepentacetic acid).

Contrast agents are typically employed to vary or alter the real or apparent response of a voxel (e.g., a volume element, representing a value on a regular grid in three dimensional space) of tissue and/or blood. Specifically, contrast agents include molecules that have and/or induce various magnetic responses to RF and magnetic excitations by the MRI system. These characteristics are generally expressed as T1, T2 and T2* relaxation times. When imaged, a molecule having a given relaxation time may appear lighter or darker than other molecules that have a shorter or longer relaxation time, thereby providing visual contrast between tissue and blood, for instance. In the case of water molecules, the contrast agent may influence the relaxation rate of the water molecule protons, thereby modifying the molecule's relaxation time. Employing SPIO, for example, may shorten the T2* relaxation time, resulting in a dark region associated with the location of the SPIO. This phenomenon is the result of the high magnetic susceptibility of the contrast medium that significantly modifies the local magnetic field.

In some procedures, the contrast agent is tissue specific and is employed to accumulate in specific regions of a patient's anatomy. For example, in the case of multiple sclerosis, gadolinium-DTPA can be administered to image break down of the blood-brain barrier, thereby indicating the severity of the disease. Similarly, in the case of injected stem-cells, iron oxide (e.g., SPIO) can be absorbed by the stem-cells prior to their injection, enabling imaging to track the location of the stem-cells and to determine whether the stem-cells have reached their target location (e.g., the heart). Although observing the region with the contrast agent may be helpful, it may be desirable to determine the quantity (e.g., amount) of a contrast agent located within a region. For example, it is useful to know if all or only some of the contrast agent has reached its target destination. Unfortunately, in an MR image the presence of the contrast agent may appear as a spot that may or may not be indicative of the quantity of the contrast agent in the region. The presence of dark spots in the image may also be indicative of inherent changes to the local magnetic field environment independent of exogenous contrast agents. These inherent changes result in a loss of spin-spin coherence over a voxel, resulting in a cancellation of the net transverse magnetization and yielding no net signal.

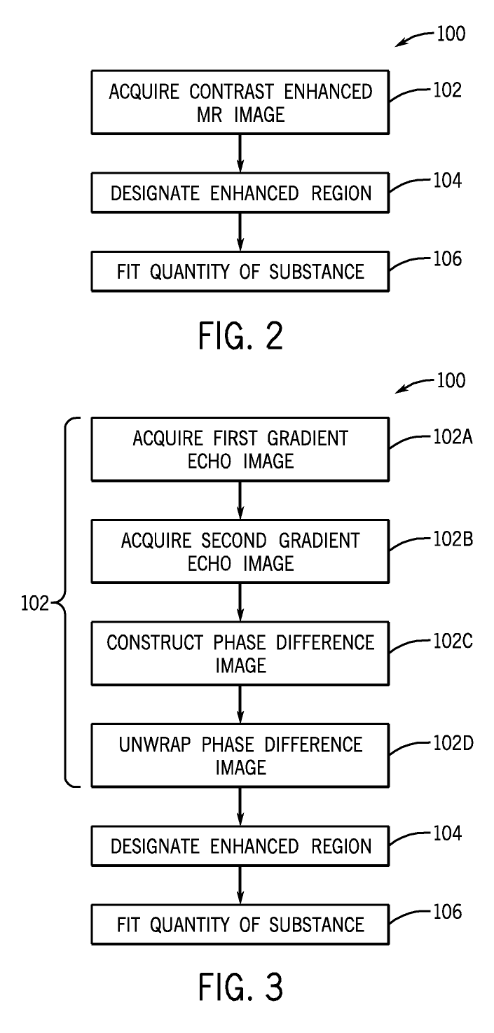

Turning now to FIG. 2, depicted is a flowchart that illustrates a method 100 of determining the quantity of a substance, such as a contrast agent, in a region of an image. The method 100 includes acquiring a contrast enhanced MR image, as illustrated at block 102, followed by designating an enhanced region, as illustrated at block 104, and fitting the quantity of the substance as illustrated at block 106. In such an embodiment a practitioner may acquire an MR image, designate a region of interest and employ the fitting algorithm(s) and techniques discussed in detail below to determine a quantity of the contrast agent.

In one embodiment acquiring a contrast enhanced MR image includes obtaining one or more MR images that include one or more enhanced regions indicative of a substance, such as a contrast agent, that delineates those regions from surrounding regions in the MR image. As discussed in further detail below with regard to FIG. 3, in various embodiments the enhanced image may include one or more of a gradient echo image, a phase difference image, and/or an unwrapped phase difference image. Other embodiments may include images that provide pertinent information for the fitting algorithm, such as phase information.

As mentioned previously, the enhanced region is generally indicative of the presence of the substance, such as a contrast agent, and it is desirable to determine the amount of the substance in the one or more enhanced regions. Selecting a location indicative of the center of the enhanced region may provide a basis for fitting the quantity of the contrast agent at that point to the effects of the resulting magnetic field at points located in the space/volume surrounding the enhanced region. Accordingly, to provide a basis for fitting the amount of the substance (block 106), designating an enhanced region (block 104) includes selecting a center of the enhanced region, in one embodiment. For example, an operator may visually inspect the image, determine the center of the enhanced region, and make a manual selection to designate the center of the enhanced region for use in the fitting algorithms. In another embodiment, selecting the enhanced region (block 104) may be automated. For example, a separate algorithm and/or image processing technique, such as thresholding and/or BLOB (binary large object) analysis, can be employed to determine the center or corresponding region of interest of the enhanced region.

Designating the enhanced region (block 104) includes the selection of more than one enhanced region, in some embodiments. For example, in one embodiment, regions surrounding the enhanced region are selected to perform the best-fit analysis. Similarly, in one embodiment that includes multiple enhanced regions, designating the enhanced region includes selecting two or more of the enhanced regions believed to include the substance (e.g., the contrast agent). As will be appreciated, where more than one enhanced region exists in the image, designating more or all of the enhanced regions may enable the fitting to more accurately determine the quantity of the contrast agent at one or more enhanced regions. Some embodiments may include selecting multiple enhanced regions and locations. For example, the location of the dipole center can be varied in location and/or orientation. This may be performed with multiple enhanced regions identified in a single run of the fitting or may be performed in a series of fitting performed one after another. In such an embodiment, the enhanced regions and location that includes the best least-squares fit (see the discussion block 104 below) may be employed to determine the quantity and location of the contrast agent.

Based on the enhanced regions identified at block 104, the quantity of the substance in the enhanced region(s) is determined by fitting. In one embodiment, as discussed in greater detail below with regard to FIG. 3, fitting may include employing a least-square fit that is based on the identified enhanced regions 104. For example, in one embodiment, the identified enhanced regions include several varying phase distributions that are fit to an expression of the expected spatially varying phase distribution from a center of the substance/contrast agent. The fitting may yield a result indicative of the quantity (e.g., amount) of the substance in the region.

Turning now to FIG. 3, an embodiment of the method of FIG. 2 is illustrated. More specifically, the illustrated embodiment includes details of the step of acquiring the contrast enhanced MR image (block 102). For example, the method 100 includes acquiring a first gradient echo image and a second gradient echo image, as illustrated at blocks 102A and 102B, respectively. The echo images are generally derived from a pulse sequence in which the phase spatial variation is proportional to the echo time and the magnetic field in proximity of the agent. In one embodiment, acquiring the first and second gradient echo images (blocks 102A and 102B) includes acquiring a first gradient echo image having a first echo time (TE) (e.g., TE=10 milliseconds (ms)) and a second gradient echo image having a second TE (e.g., TE=25 ms). The first gradient echo image and the second gradient echo image are obtained via separate image sequences, in one embodiment. For example a first image sequence is employed to acquire the first gradient echo image followed by a second image sequence to acquire the second gradient echo image. In another embodiment, the first gradient echo image and the second gradient echo image are obtained from a dual echo imaging sequence. The dual echo imaging sequence enables both of the first and second gradient echo images to be acquired by a single pulse sequence. As will be appreciated by one of ordinary skill in the art, the imaging sequence can include a gradient-recalled-echo pulse (GRE) sequence similar to those discussed above, or any variation thereof. Generally, the pulse sequence is designed such that at the echo time, the signal phase is affected by the Larmor frequency of the local tissue.

In some embodiments, acquiring a first gradient echo image and a second gradient echo image (blocks 102A and 102B) includes selecting echo times that are multiples of the precessional time for fat and water such that the fat and water signals are in phase. For example, in one embodiment, the phase difference image includes an image derived from one or more acquisition pulse sequences wherein the phase difference image includes an image derived from one or more acquisition pulse sequences configured such that a phase difference between fat and water at a first echo time is substantially the same as the phase difference between fat and water at a second echo time. Such echo times can be based on the characteristics of the MR system used to acquire the image. In some embodiments, the MR system can automatically determine echo times to place the precessional time for fat and water in phase. In another embodiment, the echo times are selected to eliminate phase errors or phase due to the chemical shift effects of fat relative to water spins. For example, the echo times are selected such that the precessional times of fat and water are out of phase by the same (or a comparable) amount. In one embodiment, the phase difference image includes an image derived from one or more acquisition pulse sequences configured such that there is substantially no phase difference between fat and water at a first echo and a second echo time. Once again, embodiments of these techniques, and those discussed below, may include the first gradient echo image and the second gradient echo image obtained via separate image sequences or a dual echo imaging sequence.

Further, in some embodiments, acquiring a first gradient echo image and a second gradient echo image (blocks 102A and 102B) includes acquiring images in a manner to compensate (or account) for motion (e.g., flow). For example, in one embodiment, the acquisition pulse sequence(s) is constructed such that the second echo has the same first gradient moment as the first echo has. This can provide first order flow (velocity) compensation of the second echo relative to the first echo. For example, in one embodiment, the image acquisition sequence generates a first echo at a first echo time (TE1). Since the moment of the gradients about the second echo is the same as about the first echo, the phase at a second echo time (TE2) due to flowing spins approximates that of the first echo time (TE1).

Another embodiment includes utilizing an acquisition pulse sequence(s) where the first gradient moment is nullified relative to the first echo and the second echo. In other words, dephasing of intravoxel flow may be minimized by flow compensating the first and second echo signals (e.g., zero first gradient moment). For example, in one embodiment, the acquisition pulse sequence is constructed such that the first gradient moments in both the first echo and second echo are zero. Such an embodiment may improve the image-to-noise ratio (S/N).

The depicted method 100 also includes constructing a phase difference image, in one embodiment, as illustrated at block 102C. As will be appreciated by one of ordinary skill in the art, in one embodiment, the phase difference image can be acquired from the difference between multiple gradient echo images, such as the first gradient echo image and the second gradient echo images acquired at blocks 102A and 102B, respectively. In a phase difference image, extraneous spatial phase variations may cancel, enabling more accurate fitting of the amount of the substance. For example, where a single gradient-recalled echo image may contain spatially varying phase from the contrast agent (e.g., SPIO), eddy currents, echo mis-centering, and inhomogeneity of the magnetic moments from other sources, a phase difference image may remove residual phase artifacts that are not attributed to the presence of the contrast agent.

In one embodiment, constructing the phase difference image (block 102C) includes accounting for spatially varying residual background phase. An embodiment includes a second order phase correction performed on the phase difference image after low pass filtering and masking the region where the contrast agent may be present. For example, where the varying residual background phase from the phase difference image is present, one embodiment includes generating a low pass filtered phase difference image (e.g., convolving the phase difference image with a low spatial frequency filter), masking out the region where the contrast agent (e.g., SPIO) concentration is believed to be present (e.g., an organ of interest) and its immediate surroundings where the phase may be significantly affected by the agent, fitting the resulting low-pass filtered and masked phase difference image to a spatially dependent phase difference (e.g., filtering to determine the coefficients of a second order spatially varying function (phase map) that models phase variation from eddy currents and/or echo mis-alignments), and subtracting out the spatially dependent phase difference (e.g., the spatially varying function (phase map) determined previously) from the resulting phase difference image.

For example, in one embodiment, the resulting phase difference image of a two-echo gradient acquisition can be expressed as:

arg(S(\vec{r}t=TE₁)S*(\vec{r},t=TE₂))=φ_{S}(\vec{r})−φ_{S}′(\vec{r})+φₑ(\vec{r})−φₑ′(\vec{r})+_{f}(\vec{r})−φ_{f}′(\vec{r}) [1]

Where φ_{S}(\vec{r}), φ_{S}′(\vec{r}), φₑ(\vec{r}), φₑ′(\vec{r})φ_{f}(\vec{r}), and φ_{f}′(\vec{r}) represent the phase accumulation for the first and second phase images due to contrast material (S), eddy currents (including concomitant gradient effects) (e), and flow (f), respectively, where * indicates a hermitian conjugate operator. The spatially dependent phase difference due to eddy currents, diamagnetic suseptibiltiy from patient body habitus, and concomitant gradient effects is expressed as:

Δφₑ(\vec{r})=φₑ(\vec{r})−φₑ′(\vec{r}) [2]

where Δφₑ(\vec{r}) is the spatially dependent phase difference. Further, Δφₑ(\vec{r}) can be expressed as a second order spatially varying function:

φₑ(\vec{r})−φₑ′(\vec{r})=a₀′+a₁₁′x+a₁₂′y+a₁₃′z+a₂₁′x²+a₂₂′y²+a₂₃′z²+a₂₄′xy+a₂₅′yz+a₂₆′xz [3]

where aₙ′ are those from the difference between the first and second echo times. In such an embodiment, fitting the image to a spatially dependent phase difference includes fitting the resulting low-pass filtered and masked phase difference image to determine the coefficients of equation [3]. Accordingly, subtracting out the spatially dependent phase difference from the resulting phase difference image includes subtracting equation [2] or equation [3] from equation [1].

In some embodiments, the techniques discussed above are performed separate from or in addition to the flow compensation discussed previously with regard to block 102A and blocks 102B. Combining flow compensation (e.g., gradient moment nulling in the pulse sequence) and a background phase correction (i.e., correcting the phase difference image to account for spatially varying residual background phase) can yield a phase difference image that is directly related to the amount of susceptibility contrast agent. Although the techniques for correction can be used in combination, in embodiments, each of the corrections techniques may used alone or in any combination to yield an image directly related to the amount of the agent.

Further, the depicted method 100 includes unwrapping the phase difference image, in one embodiment, as illustrated at block 102D. As will be appreciated by one of ordinary skill in the art, unwrapping the phase difference image may be accomplished by one or more standard unwrapping techniques. For example, the phase difference image may be unwrapped using the Goldstein method, in one embodiment. Other embodiments may include the use of other unwrapping methods, such as the method of Buckland and Huntley ([1] Huntey J. M. “Noise-immune phase unwrapping algorithm” Appl. Opt. 28 3268-3270 (1989) [2] Buckland J. R., Huntley J. M., Turner S. R. E “Unwrapping noisy phase maps using a minimum-cost-matching algorithm” Appl. Opt. 34(25) 5100-5108 (1995)), or eg Chavez, S.; Qing-San Xiang; An, L., “Understanding phase maps in MRI: a new cutline phase unwrapping method,” Medical Imaging, IEEE Transactions on, vol. 21, no.8, pp. 966-977, August 2002, or any combination thereof. Further, in certain embodiments, unwrapping may be performed at various steps within the method 100. For example, in the illustrated embodiment, unwrapping (block 102D) is performed after constructing the phase difference image (block 102C). However, in another embodiment, unwrapping (block 102D) is performed before constructing the phase difference image (block 102C), for instance.

Similar to the technique discussed with regard to FIG. 2, the method includes designating one or more enhanced regions (block 104) and fitting the amount and/or the location of the substance, and/or the direction of the magnetization of the substance (block 106). Fitting generally includes comparing the observed phase to a predicted phase and selecting the amount of substance that makes the predicted phase substantially match the observed phase. In one embodiment, fitting the amount of the substance to a location includes fitting the amount of the substance (e.g., contrast agent) to an unwrapped phase difference image generated at block 102D. More specifically, in one embodiment, fitting the quantity of the substance (block 106) includes selecting, based on the image, a dipole center of the substance, and employing a least-square fit of the dipole center based on the unwrapped phase difference image and the spatially varying phase distribution from the center. Accordingly, the amount of the substance can be associated with a position at or near the selected dipole center. This may provide an indication how much contrast agent has accumulated in a particular region of a patient, for instance.

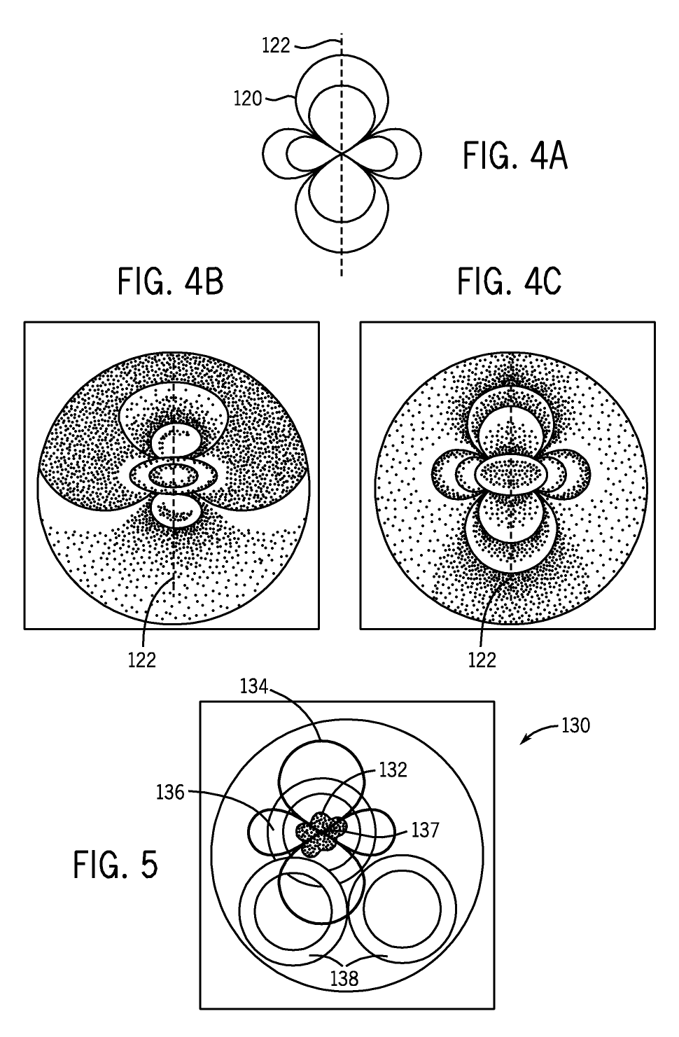

Such a technique is possible due to the characteristics of MR images and the substances being imaged. Specifically, in the case of a super paramagnetic contrast agent, such as SPIO, the contrast agent actually changes the signal phase of nearby tissue. The change may be less significant at greater distance from the center of the contrast agent. Accordingly, the magnetic field of the contrast agent affects image information (e.g. pixel values) indicative of signal phase. For example, as depicted and discussed in further detail below with regard to FIG. 4, the gradients may appear as bands that appear similar in color/shade for a given phase, and they are indicative of the contour of the phase change across the image, presumably due to one or more magnetic fields generated by the contrast agent. In one embodiment, the phase change is identified by counting the number of bright (or dark) bands.

Turning now to exemplary images, FIGS. 4A-4C illustrate a dipole field pattern and phase images exhibiting a dipole pattern. More specifically, FIG. 4A illustrates a dipole pattern 120 having a Z-axis 122. The dipole pattern is illustrated in the two-dimensional image plane for simplicity; however, such a dipole 120 typically exhibits a three-dimensional pattern that may be represented by the revolution of the illustrated pattern about the z-axis. FIG. 4B is a plot of a phase image acquired from a SPIO-laden silicone bead disposed in an agarose gel, wherein the phase is acquired with a 7.5 ms TE. FIG. 4C is a plot of a phase difference image acquired from the same SPIO laden silicone bead disposed in an agarose gel, wherein the phase difference is taken between a 7.5 ms TE and a 23.5 ms TE. As illustrated, the images of FIG. 4B and FIG. 4C both illustrate a pattern that is representative of the phase contour from the center of the SPIO-laden region and that is closely matched to the typical dipole pattern illustrated in FIG. 4A. More specifically, the varied intensity of the bands indicates the change in frequency (e.g., from light (0 degrees phase difference) to dark (e.g., 360 degree phase difference)) that is a result of the dipole magnetic field. As will be appreciated, certain embodiments, including those discussed in detail describe the use of a phase difference image, such as that of FIG. 4C for implementation of the fitting algorithm. However, other embodiments may employ the use a single phase image, such as that of FIG. 4B. The information presented in the phase image may be extracted for use in the fitting algorithm as discussed below. For example, certain points or regions (e.g., enhanced regions) of the patient 22 may be associated with points/regions/pixels in the phase image, and the phase information at the points/regions/pixels in the phase image used in the fitting algorithm to determine the quantity of the substance (e.g., contrast agent) in the enhanced region.

Turning now to the algorithms used in fitting the amount of the substance, where the contrast agent is assumed to be a point dipole, least-square fitting can estimate the location and magnitude of the dipole, and therefore the amount of the contrast agent (e.g., SPIO). In one embodiment, the spatially varying phase distribution from the dipole center (e.g., the selected enhanced region) of the contrast agent is expressed by the following equation:

$$Δϕ ( r ) = m γ \frac{( 3 {cos}^{2} θ - 1 )}{{\vert r \vert}^{3}} Δ TE \qquad [ 4 ]$$

where m is the magnetization of the dipole, y is the gyromagnetic ratio, θ is the azimuth (e.g., the angle between the line from the dipole center to the point (pixel) under consideration) and r is the radial distance of the point (pixel) from the dipole center. Accordingly, various points having a given θ and distance (r) from the dipole center are associated with a given phase (e.g., by way of the information extracted from the phase image). Based on the known azimuth (θ), distances (r) and phases, equation [4] is employed to solve for the magnitude of the magnetic dipole (m), and based on the magnetic dipole (m) the quantity of the substance (e.g., the contrast agent) is determined.

Although the illustrated embodiments depict the selection of multiple points in a two-dimensional plane, a similar embodiment can include the selection of one or more points in a three-dimensional space surrounding the dipole. As discussed previously, the dipole generally exhibits a magnetic field in at least three-dimensions, and, therefore, points can be chosen from the three dimensional space as long as their relationship (e.g., orientation—distance and angle) to the dipole is known and/or determinable. The fitting of points in three dimensions can be accomplished in a similar manner as the method discussed above with regard to points taken from a two-dimensional image. For example, knowing the phase and distance of one or more points in the volume surrounding the enhanced region, the quantity can be estimated based on the same equation [4] relating distance, phase, and the magnetic moment.

Turning now to FIG. 5, an exemplary image 130 is illustrated. More specifically, FIG. 5 depicts an MR image of a rat leg injected with approximately 3 μg (microgram) of SPIO iron. In one embodiment, the image 130 includes an enhanced MR image that is used by an operator to designate one or more regions of interest for used by the fitting algorithm in the determination of the quantity of the substance. In one embodiment, similar to that discussed above, an operator selects the dipole center of the contrast agent based on visual inspection of the image 130. For example, the operator may observe a dark spot within the image 130 and select the approximate center of the spot. In the illustrated embodiment, the image 130 includes a dark spot 132 that is indicative of the point dipole created by the SPIO concentration. Also present on the image 130 is an optional operator-located icon 134 which may be an arrow, box, or other marking and is shown in FIG. 5 as a shape representative of the dipole magnetic field pattern) and spherical shell 136 having its center aligned with the approximate dipole center 137 of the dark spot 132. Although the illustrated embodiment includes the spherical shell 136 located at the center 137 of the dark spot 132, other embodiments may include the spherical shell 136 off-center from the center 137. Further, the spherical shell 136 may be located in a region that is representative of a volume proximate the surface of the body (e.g., where fitting may include regions external to the body that are primarily composed of air). The operator may adjust the size and/or shape of the spherical shell 136 to modify the fitting region. Similar to the operator selecting the dipole center 137 of the contrast agent, the operator may select the regions surrounding the dark spot to be used in the least-square fitting. For example, the operator may select one of more regions in the image space surrounding the contrast agent, thereby providing a basis for the least-squares fitting. The remaining two operator located control regions 138 are representative of the regions selected by an operator for performing the fit. One or more optional control regions 138 that the operator has identified as lacking agent, may be employed to verify the precision and sensitivity of the method.

Based on the approximate dipole center 137 selected by the operator, and the phase characteristics of the regions surrounding the dark spot 132 (e.g., the contrast agent), a least-square fitting is accomplished using the relationship expressed in equation [4] and the one or more selected enhanced regions. For example, based on the previously discussed fitting technique and the spherical shell 136 and control regions 138, the image 130 includes of a rat leg injected with 3 μg (micrograms) of SPIO and the circle 136 located over the dark spot 132 was estimated to have approximately 2.9 μg of SPIO. As will be appreciated, the difference between the 3 μg of SPIO injected and the estimated 2.9 μg of SPIO can be attributed to injected SPIO that is not concentrated in the control region 136, but is instead dispersed elsewhere in the rat leg.

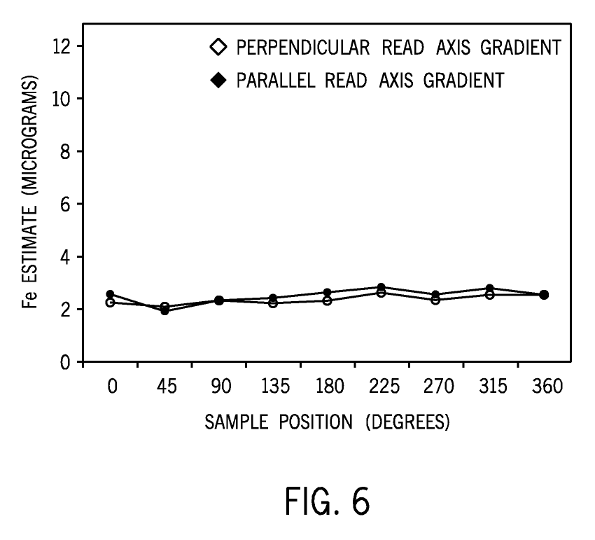

FIG. 6 is a graph that illustrates additional estimates of the SPIO contained in the leg illustrated in FIG. 3 wherein the phase difference images (first gradient echo image and second gradient echo image) were taken at a TE of 9.8 ms and 25 ms respectively. Data were collected with the read and phase encode axes interchanged and with the sample rotated to nine different orientations, from 0 degrees to 360 degrees in steps of 45 degrees. Images made in sagittal orientation (to scanner) with read gradient axis parallel to BO are represented by solid symbols and with read gradient axis perpendicular are represented by the open symbols. Phase difference fitting estimates were approximately 2.48+/−0.26 μg.

Although the previous discussion has focused on determining the quantity of the substance in a region if interest, determining the sensitivity of the technique may be useful in some applications of the technique. For example, in addition to the determination of the quantity of the substance, the sensitivity of the previously discussed techniques can be expressed in relation to the sensitivity of regions proximate to the labeled tissue (e.g., the tissue containing the contrast agent) and the regions surrounding the labeled tissue. For example, if the magnetic properties of the contrast agent and of labeled and surrounding tissues are known, sensitivities of T₂* and phase methods can be predicted.

According to the T₂* Method the signal from the labeled tissue can be expressed as:

S=exp(−TE(R₂₀*+cr₂*)) [5]

where relaxivity is r₂*, and R₂₀* is inverse of unenhanced T₂*. In such an embodiment sensitivity may be defined as the derivative of signal with respect to c, the agent concentration, at the optimum TE and at a vanishing concentration. The derivative of sensitivity with respect to TE shows the optimum TE is 1/R₂₀*, T₂*. At TE=1/R₂₀* and c=0, the sensitivity is:

$$\vert \frac{d S}{d c} \vert = \frac{{r}_{2}^{*}}{{eR}_{20}^{*}} \qquad [ 6 ]$$

Note, the units are 1/mM/s for r₂* and 1/s for R₂₀*. Thus, sensitivity units are liters/mmole iron.

According to the Phase Method the same procedure followed for magnitude and T2*-weighting can be applied to the phase of the surrounding tissue. The signal of the surrounding tissue can be expressed as:

S=exp(−TE(R₂₀*+iω))=exp(−TE(R₂₀*+i4πγI/3)). [7]

The change in Larmor frequency, ω, caused by SPIO in the nearby, labeled region is proportional to the amount of SPIO present. The latter expression in Eq. [7] applies to the equator of a spherical distribution with concentration I in emu/cm³. As above, 1/R₂₀* is the optimum TE. Sensitivity based on magnetic units instead of moles is:

$$\vert \frac{d S}{d I} \vert = \frac{4 πγ}{3 {eR}_{20}^{*}} \qquad [ 8 ]$$

Expressing concentration in mM with the conversion factor k facilitates comparison with the T₂* method. Rewriting Eq. [7] and Eq. [8]:

$$\begin{matrix}\begin{matrix}S = exp ( - TE ( {R}_{20}^{*} + iω ) ) \\ = exp ( - TE ( {R}_{20}^{*} + i4πγ ck / 3 ) )\end{matrix} & [ 9 ] \\ \vert \frac{d S}{d c} \vert = \frac{4 πγ k}{3 {eR}_{20}^{*}} & [ 10 ]\end{matrix}$$

Here k=msat MW 10⁻⁶ with msat in emu per gram of iron. MW the molecular weight of iron and the 10⁻⁶ converts liters to cm³ and mmoles to moles.

While only certain features of the invention have been illustrated and described herein, many modifications and changes will occur to those skilled in the art. It is, therefore, to be understood that the appended claims are intended to cover all such modifications and changes as fall within the true spirit of the invention.

1. An imaging method, comprising:
accessing or acquiring a phase image including a contrast agent enhanced region; and
determining a quantity of a contrast agent based on the phase image.

2. The method of claim 1, wherein the phase image comprises a phase difference image.

3. The method of claim 2, wherein the phase difference image is acquired from a multi-echo gradient-recalled echo acquisition pulse sequence.

4. The method of claim 2, wherein the phase difference image is derived from two or more gradient-recalled echo images.

5. The method of claim 4, wherein each of the gradient-recalled echo images comprises an image derived from a pulse sequence comprising variation of phase proportional to the echo time and a magnetic field proximate the contrast agent.

6. The method of claim 2, wherein the phase difference image comprises an image derived from one or more acquisition pulse sequences configured such that a phase difference between fat and water at a first echo time is substantially the same as the phase difference at a second echo time.

7. The method of claim 2, wherein the phase difference image comprises an image derived from one or more acquisition pulse sequences configured such that there is substantially no phase difference between fat and water at a first echo and a second echo time.

8. The method of claim 2, wherein accessing or acquiring the phase image comprises compensating for motion.

9. The method of claim 8, wherein compensating for flow motion comprises employing one or more acquisition pulse sequences configured to null a first gradient moment relative to a first echo.

10. The method of claim 8, wherein compensating for flow motion comprises employing one or more acquisition pulse sequences configured to null a first gradient moment relative to a first echo and a second echo

11. The method of claim 1, comprising correcting the phase difference image to account for spatially varying residual background phase.

12. The method of claim 11, wherein correcting the phase difference image to account for spatially varying residual background phase, comprises:
low pass filtering the phase difference image;
masking out a region where the contrast agent is believed to be present and where a phase is significantly affected by the contrast agent;
fitting the resulting low-pass filtered and masked phase difference image to a spatially dependent phase difference; and
subtracting out the spatially dependent phase difference from the resulting low-pass filtered and masked phase difference image.

13. The method of claim 1, comprising designating a dipole center positioned at an enhanced region of the contrast agent,
predicting the phase or phase difference at one or more voxels outside of the enhanced region; and
measuring the phase or phase difference at the one or more voxels outside of the enhanced region.

14. The method of claim 13, wherein fitting the quantity of the contrast agent comprises employing a least squares fitting of the one or more voxels.

15. The method of claim 14, wherein employing a least squares fitting is based on a spatially varying phase distribution expressed or predicted by:
$$Δϕ ( r ) = m γ \frac{( 3 {cos}^{2} θ - 1 )}{{\vert r \vert}^{3}} Δ TE$$
where m is a magnetization dipole, γ is a gyromagnetic ratio, θ is an azimuth angle and r is a radial distance.

16. A method of estimating a quantity of a contrast agent, comprising:
(a) constructing a phase difference image based on first and second images;
(b) unwrapping the phase difference image to generate an unwrapped phase difference image;
(c) designating a dipole center of a depicted contrast agent in the unwrapped phase difference image;
(d) designating one or more surrounding regions of interest, wherein the one or more surrounding regions of interest do not overlap the depicted contrast agent; and
(e) estimating the quantity of the contrast agent based on the identified dipole center and the one or more surrounding regions of interest.

17. The method of claim 16, wherein the phase difference image comprises a three-dimensional image.

18. The method of claim 17, wherein the one or more surrounding regions of interest comprises one or more locations located in a three dimensional space surrounding the dipole center.

19. The method of claim 16, wherein estimating the quantity of the contrast agent comprises fitting the predicted phase map to the observed phase or phase difference image.

20. The method of claim 19, wherein fitting comprises varying the designated location of the dipole center.

21. A method, comprising:
estimating a quantity of a contrast agent depicted in an MR image based on the phase values observed in a region of the MR image that surrounds the depicted contrast agent.

22. The method of claim 21, comprising selecting a first location indicative of the center of the contrast agent, and selecting one or more second locations located a distance from the first location.

23. The method of claim 22, wherein the one or more second locations in a volume surrounding the contrast agent.

24. A computer readable medium storing a computer program for determining the quantity of a substance in a region, the program constructed and arranged to:
access or acquire a contrast enhanced image;
execute a fitting algorithm based on data contained in the contrast enhanced image; and
determine the quantity of the substance based on the output/result/outcome of the fitting algorithm.

25. The computer program of claim 19, wherein the contrast enhanced image comprises a phase difference image.
